(19) United States
(12) Patent Application Publication
(10) Pub. No.: US 2011/0159126 A1
(43) Pub. Date: Jun. 30, 2011

(54) Composition and method for exfoliating skin

(76) Inventor: Paul Ling Tai, Chelsea, MI (US)

(21) Appl. No.: 12/928,158

(22) Filed: Dec. 6, 2010

Related U.S. Application Data
(62) Division of application No. 12/317,679, filed on Dec. 29, 2008, now abandoned.

(51) Int. Cl. A61K 36/42

(52) U.S. Cl. 424/758

(57) ABSTRACT

Disclosed is an exfoliating composition and method for removing dead skin cells from the surface of human skin. The composition is a physiologically effective amount of ground up cucumber or cucumber extract in a suitable carrier. The carrier can be water, alcohol, or mixtures thereof. The method is to administer a physiologically effective amount of the composition to an individual seeking to exfoliate skin. The composition is rubbed into the skin, and loosens dead cells, which are then physically removed from the surface of the skin.

This application is a Divisional of Ser. No. 12/317,679, filed Dec. 29, 2008, assigned to Examiner Melenia Lee McCormick, Art Unit 1655.

### BACKGROUND OF THE INVENTION

(a) Field of the Invention

This invention relates to a composition and method for exfoliating skin on a human being. More particularly, it relates to a composition containing a cucumber extract, that is dissolved in or otherwise incorporated into a suitable carrier, and to a method of exfoliating skin from the human body which involves rubbing a ground up cucumber or a composition containing the cucumber extract onto skin, and thereafter removing the accumulated dead skin cells from the surface thereof.

The skin is the protective barrier for the human body. It protects the interior of the body from physical injury, i.e., trauma, and biological injury from such factors as bacteria, viruses, fungi, noxious gases, and the like. The skin of the human body comprises the dermis, and the epidermis. The epidermis is the top most layer of the skin. It is about 0.1 to 1.5 millimeters thick. It is made up of five layers. From bottom to top, the layers are: basal cell layer, the squamous cell layer, the stratum granulosum, the stratum lucidum, and the stratum corneum. These layers continually rebuild the surface of skin from within, maintaining the skin's strength and helping avoid wear and tear on it. The process begins with the basal cell layer. The basal cells eventually work their way upward, being ultimately transformed into the stratum corneum, or layer of dead cells. The stratum corneum, or horny layer, is normally made up of 10 to 30 layers of dead cells. These cells are constantly being replaced by new cells from below. It is estimated that in the average adult it takes about a month for the stratum corneum to be completely replaced by new cells. As human beings age, the replacement time increases from about a month to 120-150 days. The longer it takes to replace the dead cells, the more layers of dead cells accumulate. Thus in older individuals, the skin accumulates up to 150 layers of dead cells. Moreover, as aging takes place, the appearance of the skin changes. Skin with the optimum stratum corneum layer of 10 cells is shiny and provides a youthful appearance, while skin with 150 layers of dead cells has a grayish and dull look. New skin has the appearance and cellular quantity of younger skin with higher collagen and elastin content.

(b) Description of Related Art

It is desirable, therefore, to eliminate excess layers of dead skin from the skin surface in order to provide a shiny skin with a youthful appearance. The process of removing dead skin cells from the skin surface is called exfoliation. Currently, human skin is exfoliated in a number of different ways. Among the techniques are: mechanical scrubbing using sand, diamonds, stones, or hard particles of any kind. Lasers are also used to burn the dead skin off, and chemical peels are very popular. There are a number of peels used, including acid peels, base peels, and enzyme peels, all of which are used to digest the dead skin cells. Examples of various exfoliating compositions and methods are disclosed in U.S. Pat. Nos. 6,416,769; 6,036,966; 5,505,948; 6,645,514; and 10,860,726.

There are several problems associated with each of the above identified methods of dead skin removal. Chief among these problems are that the processes cause burns on the skin, redness, irritation, inflammation and even pain, and there may be a recovery time up to 30 days.

The search for a safe, painless, non-irritating and effective composition and method for exfoliating dead skin cells has been ongoing.

### BRIEF SUMMARY OF THE INVENTION

One embodiment of the present invention is an exfoliating composition, i.e., a composition for removing dead cells from the surface of skin. The composition comprises an extract of cucumber incorporated into a suitable carrier. The carrier can be water or alcohol based, or a mixture thereof. The carrier can be in the form of a liquid, paste, cream, lotion or gel. A gel is preferred for reasons apparent below.

Another embodiment of the invention includes a method of exfoliation, i.e., removing dead skin cells from the surface of human skin. The method comprises applying a physiologically effective amount of the composition as described above containing a cucumber extract to the topmost surface of skin, rubbing it into the skin for a sufficient period of time to allow it to loosen dead cells, and subsequently removing the cells by physical means.

As used herein, the term “exfoliation” refers to a detachment and shedding of superficial cells of an epithelium or from any tissue surface. Tissue surfaces include but are not limited to facial skin, skin on the soles of feet, knees, elbows, legs, arms, and other skin areas. Exfoliation also includes detachment or shedding of calloused skin, such as skin on the sole and heel of a foot, skin on an elbow and callouses on other parts of the body.

The term ‘pharmaceutical” refers to a formulation administered to the skin which renders a benefit or an effect for treating or preventing an abnormal biological condition.

The term “physiologically effective” refers to that amount of the composition of the invention, which when applied to the surface of the skin, is effective to loosen the bonds between dead skin cells, enabling them to be removed.

### DETAILED DESCRIPTION OF THE INVENTION

One embodiment of the present invention includes a non-irritating exfoliation composition. The exfoliation composition of the present invention includes creams, gels, foams and pastes. The formulation includes a diluent such as water, aqueous alcohol, glycol or other inactive carrier which includes up to about 99 percent ground cucumber or cucumber extract defoliating material. The composition includes, for some embodiments, carrier, excipient, or vehicle ingredients such as, for example, water, gels, carbomers, acetone, ethanol, ethylene glycol, proplylene glycol, butane-1,3 diol, isopropyl myristate, isopropyl palmitate, mineral oil, and mixtures thereof to form lotions, tinctures, creams, emulsions, gels, or ointments which are non-toxic and pharmaceutically, cosmetically or dermatologically acceptable. Additionally, moisturizers or humectants are added to the present composition, if desired. The composition can also include materials such as vitamins, peptides, minerals, microminerals, exfoliates, hydration, other proteins, collagens, and the like.

In addition to the diluent, formulations of the composition also, for some embodiments, include other standard adjuvants such as an emollient, moisturizer, emulsifier, neutralizer, coloring agent, UV absorber or filter, preservative and or gelling agent. When employed in a formulation, these adjuvants are present in amounts ranging from about 0.5% to 30%. Such standard adjuvants are described in detail in U.S. Pat. No. 7,115,251, which is incorporated herein by reference.

It is preferred that the composition be in the form of a gel. Suitable gelling agents include water soluble or colloidally water soluble polymers and include cellulose ethers, such as hydroxyethyl cellulose, methyl cellulose, hydroxypropylmethyl cellulose, polyvinylalcohol, polyquaternium-10, guar gum, hydroxypropyl guar gum and xanthan gum. Other gelling agents usable in the present invention include acrylic acid/ethyl acrylate copolymers and carboxyvinyl polymers. Also usable are maleic anhydride-alkyl methylvinylethers and copolymers, natural gums, and polymethacrylate copolymer. Other suitable gelling agents include oleogels such as trihydroxystearin and aluminum magnesium hydroxy stearate.

Another embodiment of the present invention is a method of defoliation. The method includes topically administering a pharmaceutically effective amount of the composition of the present invention to an area of the subject's skin, for a period of time sufficient to achieve epidermal exfoliation. The composition is conventionally rubbed into the skin by use of the subject's fingers. However, sponges, brushes, or other means can also be used. The period of time required can vary, depending on a number of factors, including the concentration of the ground cucumber or cucumber extract in the composition, the age of the subject, the location of the skin to be treated, the texture of the skin, and other factors.

Generally speaking, it has been found that effective results are obtained when the composition is applied, i.e, rubbed into the skin, for a period of time ranging from about 10 seconds to about 30 minutes. After sufficient period of time has elapsed, the composition is removed from the skin, along with the exfoliated skin cells.

In the preferred embodiment of the method of the invention, a gel composition containing the ground cucumber or cucumber extract is applied by a user to the skin area to be treated, and rubbed in with the user's fingers. After a period of time has elapsed, the dead skin cells become loosened from the underlying layers of skin, and are incorporated into the gel, forming balls of dead skin cells on the surface of the skin being treated. The balls of dead skin cells in the gel composition can be as much as 1 cm. in diameter. The balls of dead skin cells are then removed with a wash cloth, or by other suitable means.

It is within the purview of the skilled artisan to determine the appropriate period of time for the application of the composition of the invention to the skin, in order to obtain optimum results. The composition of the invention can be applied in a spa or salon, or administered in the home by a purchaser or end user.

While the underlying mechanism of the composition of the invention in causing exfoliation is not precisely known, it is believed that the ground cucumber or cucumber extract dissolves the substance binding the dead cells of the stratum corneum together, and therefore loosens them from the underlying layers, allowing them to be removed. Removal can be achieved means of washing the treated skin, or scrubbing, or a combination of both.

A series of tests were run to demonstrate the efficacy of the composition and method of the invention. In the tests, subjects were first exposed to an ultraviolet light machine which reveals dead skin. Dead skin under ultraviolet light has a whitish hue. The amount of dead skin was calculated for each subject to establish baseline values. The baseline value is the percentage of dead skin on the surface of the skin. The subjects were then asked to wash their faces with soap, and rinse, and the amount of dead skin remaining on their faces was again determined. Thereafter, they were asked to apply the composition of the invention to their faces, rub it in for 60 seconds with their fingers, then remove the dead skin from their faces. Thereafter, the amount of dead skin remaining on the face was again determined with the use of the ultraviolet light.

The composition applied to the skin in the tests was a gel composition The gel composition consisted of the following:

Skin Gel-Ingredient List

| Ingredients | Percentage |
|---|---|
| Carbomer 940 | 3.00% |
| Glycerin | 12.00% |
| Propylene glycol | 12.00% |
| Tocopheryl Acetate | 0.20% |
| Undecylenoyl glycine | 0.50% |
| Capryloyl glycine | 0.50% |
| Rosemary Extract | 0.01% |
| Cucumber oil | 0.10% |
| Cucumber peel extract 10:1 | 3.00% |
| Water | 68.69% |
| Total * | 100.00% |

* THE PERCENTAGES OF INGREDIENTS IN THE FORMULATION ARE PERCENTAGES BY WEIGHT

Testing modalities—UV light machine for skin evaluation
Derma View model#200
Prime Industrial Co LTD.
Facial skin image reveals white shades for dead skin

Baseline:

Subject's images were taken under the UV light machine without washing the face and the percentage of the dead skin on the face was calculated by quadratic measurement of vertical and horizontal facial from white shade areas.

Soap Test:

Subject's images taken using the UV light machine after washing the face with “Ivory” soap and water using the subjects own fingers for 60 seconds, followed by rinsing. The percentage of facial white shade areas was calculated by quadratic measurement of vertical and horizontal dimensions.

Cucumber Gel Test:

Subject's images taken using the UV light machine after washing the face with cucumber gel for 60 seconds using subjects own fingers, followed by a rinse. Percentage of facial white shade areas calculated by quadratic measurements multiplying the vertical times horizontal dimensions.

### Results

| Subject A | 62 year old, Male | |
|---|---|---|
| Baseline: | 72% Dead Skin | |
| Soap Test: | 62% Dead Skin; | 10% Dead skin removed |
| Cucumber Gel Test: | 46% Dead Skin, | 26% Dead skin removed |
| Subject B | 57 year old, Female | |
| Baseline: | 79% Dead Skin | |
| Soap Test: | 72% Dead Skin; | 7% Dead skin removed |
| Cucumber Gel Test: | 57% Dead Skin; | 22% Dead Skin removed |
| Subject C | 66 year old, Female | |
| Baseline: | 84% Dead Skin | |
| Soap Test: | 79% Dead Skin; | 5% Dead skin removed |
| Cucumber Gel Test: | 56% Dead Skin | 28% Dead Skin removed |
| Subject D | 34 year old, Female | |
| Baseline: | 69% Dead Skin | |
| Soap Test: | 66% Dead Skin; | 3% Dead skin removed |
| Cucumber Gel Test: | 51% Dead Skin | 18% Dead Skin removed |
| Subject E | 48 year old, Male | |
| Baseline: | 70% Dead Skin | |
| Soap Test: | 69% Dead Skin; | 1% Dead skin removed |
| Cucumber Gel Test: | 56% Dead Skin | 14% Dead Skin removed |
| Subject F | 42 year old, Female | |
| Baseline: | 74% Dead Skin | |
| Soap Test: | 70% Dead Skin; | 4% Dead skin removed |
| Cucumber Gel Test: | 52% Dead Skin | 22% Dead Skin removed |
| Subject G | 40 year old, Female | |
| Baseline: | 86% Dead Skin | |
| Soap Test: | 81% Dead Skin; | 5% Dead skin removed |
| Cucumber Gel Test: | 66% Dead Skin | 20% Dead Skin removed |
| Subject H | 34 year old, Female | |
| Baseline: | 68% Dead Skin | |
| Soap Test: | 65% Dead Skin; | 3% Dead skin removed |
| Cucumber Gel Test: | 48% Dead Skin | 20% Dead Skin removed |
| Subject I | 61 year old, Male | |
| Baseline: | 85% Dead Skin | |
| Soap Test: | 78% Dead Skin; | 7% Dead skin removed |
| Cucumber Gel Test: | 61% Dead Skin | 24% Dead Skin removed |
| Subject J | 48 year old, Female | |
| Baseline: | 88% Dead Skin | |
| Soap Test: | 77% Dead Skin; | 11% Dead skin removed |
| Cucumber Gel Test: | 58% Dead Skin | 30% Dead Skin removed |

It is clear from the foregoing test results that rubbing the face with the cucumber formulation set forth in paragraph [0020] for a period of 60 seconds, followed by removal thereof results in significantly more removal of dead skin cells from the face, as compared to simply washing it with soap. For example, subject A, a 62 year old man, had a baseline value of 72% dead skin on his face. Washing with Ivory soap left him with 62% dead skin on his face, while treating his face with the compositions of the invention, and in accordance with the method of the invention, left him with 46% dead skin on his face. Thus 26% dead skin was removed using the compositions and method of the invention as compared to only 10% being removed using a soap wash. Similar comparisons can be made with respect to the other examples shown.

In the exfoliation composition used in the above examples, the cucumber peel extract had a 10:1 ratio of extract to carrier, and was present at a 3% concentration by weight. Other ratio's and concentrations can be used equally well. What is important is that the total amount of ground cucumber or cucumber extract present in the composition, be present in pharmaceutically effective amounts, that is, that it be present in amounts which when applied, and rubbed into the skin, is sufficient to loosen the dead cells on the skin surface from the underlying strata, and allow them to be easily removed, .i.e. exfoliated.

The cucumber plant (Cucumis sativus) is a widely cultivated plant in the gourd family Cucurbitaceae. The fruit is roughly cylindrical, elongated, with tapered ends, and may be as large as 60 cm long and 10 cm in diameter. There are many varieties of cucumber grown around the world. Among these varieties are Armenian cucumber, English cucumber, Japanese cucumber, Kirby cucumber, Mediterranean cucumber, Persian cucumber, lemon cucumber and garden or common cucumber. Extracts of cucumber can be obtained from all of these varieties, and are effective in the compositions and method of this invention. Extracts of cucumber can be obtained from many sources, including Parchem Trading, Ltd., 415 Hugenot Street, New Rochelle, N.Y.; SPA Pantry, 485-18 South Broadway, Hicksville, N.Y. 11801; Sidney Essential Oil Co. Unit 4, 2-10 Fountain Street, Alexandria NSW 2015, Australia; Fraken BioChem co., Ltd., 2, Limen Road, Quigdao, 226024, China; Quigdao Fongta Trading Co. Ltd., 16, Beizhong Road, Qingdao, Shandong 266002, China. The extract used in the composition set forth in paragraph [0020] was obtained from Nantong Health Products, Jiagshn, China.

Although various illustrative embodiments of the composition and method of the invention have been described and shown herein, it is to be understood that the present invention is not limited to the precise embodiments described, and that various other changes and modifications may be affected therein by one skilled in the art without departing from the spirit and scope of the invention. All such changes and modifications are intended to be included within the scope of the invention as defined by the appended claims.

I claim:

1. A method of exfoliation which comprises:
(a) applying a physiologically effective amount of a composition comprising a ground up cucumber or an extract of cucumber in a suitable carrier to the surface of skin,
(b) rubbing the composition into the skin for a period of time sufficient for it to loosen dead skin cells, and
(c) removing the dead skin cells from the surface of the skin.

2. The method of claim 1 wherein the composition comprises from about 0.1% to about 99% by weight of an extract of cucumber in a suitable carrier.

3. The method of claim 2 wherein the carrier is selected from the group consisting of water, alcohol, and mixtures thereof.

4. The method of claim 3 in which the carrier is in the form of a gel.

5. The method of claim 2 wherein the extract comprises from about 3% by weight of the total composition.

6. The method of claim 1 wherein the composition is rubbed into the skin for a period of time ranging from about ten seconds to about thirty minutes.

7. The method of claim 6 wherein the composition is rubbed into the skin for a period of about 60 seconds.

8. A method of defoliating skin which comprises applying a gel composition comprising ground cucumber or an extract of cucumber in a suitable carrier to the skin area to be defoliated, rubbing the composition into the skin with fingers for a sufficient period of time for the composition to loosen dead skin cells from the underlying strata of skin, forming gel balls of dead skin cells on the surface of the skin, and subsequently removing said gel balls from the surface of the skin.
